(19) United States
(12) Patent Application Publication
(10) Pub. No.: US 2007/0031377 A1
(43) Pub. Date: Feb. 8, 2007

(54) Treatment of type 1 immune response-mediated inflammatory lung disease by modulation of ifn-gamma activity

(76) Inventors: Stany Depraetere, Oudenaarde (BE); Katrien Lorre, Ghent (BE); Marie-Ange Buyse, Merelbeke (BE)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(21) Appl. No.: 10/550,175

(22) PCT Filed: Mar. 26, 2004
(86) PCT No.: PCT/EP04/50375

§ 371 Date: Nov. 10, 2005

Related U.S. Application Data
(60) Provisional application No. 60/461,687, filed on Apr. 9, 2003.

(30) Foreign Application Priority Data
Mar. 28, 2003 (EP) 03447070.8

(51) Int. Cl. A61K 38/21; A61K 39/395; C07K 16/24

(52) U.S. Cl. 424/85.6; 424/145.1; 530/388.23

(57) ABSTRACT

The present invention relates to preventing, or treating and/or reducing the severity or progression of Type 1 immune response-mediated inflammatory lung disease. More particularly, the present invention provides a method for preventing or treating chronic obstructive pulmonary disease (COPD), severe asthma, sarcoidosis, berylliosis or cystic fibrosis by neutralizing or reducing IFNγ bioactivity which can be achieved either by in vivo administration of IFNγ neutralizing molecules or by in vivo immunization with pharmaceutical compositions comprising immunogenic IFNγ proteins or IFNγ-derived (poly)peptides or their corresponding nucleic acid sequences.

### FIELD OF THE INVENTION

The present invention relates to preventing, or treating and/or reducing the severity or progression of Type 1 immune response-mediated inflammatory lung disease. More particularly, the present invention provides a method for preventing or treating chronic obstructive pulmonary disease (COPD), severe asthma, sarcoidosis, berylliosis or cystic fibrosis by neutralizing or reducing IFNγ bioactivity which can be achieved either by in vivo administration of IFNγ neutralizing molecules or by in vivo immunization with pharmaceutical compositions comprising immunogenic IFNγ proteins or IFNγ-derived (poly)peptides or their corresponding nucleic acid sequences.

### BACKGROUND ART

The present invention relates to preventing the onset of symptoms, treating and/or reducing the severity or progression of COPD and other Type 1 immune response-mediated (T1) inflammatory lung diseases such as, but not limited to, severe asthma, sarcoidosis, berylliosis, and cystic fibrosis. T1 inflammatory lung diseases are characterized by a Type 1 immune response mediated by T helper-1 cells (CD4+) and T cytotoxic-1 cells (CD8+) and by increased production of interferon gamma (IFNγ), tumor necrosis factor (TNF), and interleukin-2 (IL-2). T1 cytokines evoke cell-mediated immunity characterized by prominent lung tissue infiltration of macrophages, neutrophils, and T-cells.

Severe Asthma

Asthma is a disease of the respiratory system characterized by hyper-responsiveness to bronchoconstricting stimuli, inflammation, and changes in respiratory epithelium Asthmatic patients in whom the disease process is either refractory to therapy or requires persistent use of high dose systemic anti-inflammatory corticosteroids in order to maintain reasonable control of symptoms are referred to in literature as patients suffering from severe or irreversible or refractory asthma (Kaplan N. M. et al., 2000). Patients with severe persistent asthma have continual symptoms, frequent exacerbations, frequent nighttime symptoms and evidence of severe obstructive lung disease on pulmonary function testing (Forced Expiratory Volume in one second: FEV1<60%). The narrowing of airways causes ventilation perfusion imbalance, lung hyperventilation, and increased work of breathing that may lead to ventilatory muscle fatigue and life-threatening respiratory failure (Papiris S. et al., 2002). WO 01/34180 by Block Lutz-Henning describes the use of IFNγ for the treatment of severe asthma broncliale. New effective treatments are needed for this subpopulation of asthmatic patients (Stirling R. G. and Chung K. F., 2001).

Increased numbers of neutrophils are observed in the lung mucosa and the bronchoalveolar fluid of severe asthmatics (Wenzel et al., 1997). Eicosanoid mediators such as thromboxane and leukotriene B4 are also high in the lung tissue of these patients. No difference in eosinophil concentration is observed in BAL fluid derived from healthy controls or from severe asthmatics.

Severe asthma is differentiated from mild/moderate asthma by distinct inflammatory processes involving varied cytokine expression profiles and/or effector cells. In contrast to severe asthmatics, mild or moderate asthmatics show enhanced concentrations of eosinophils in their lungs and a Type 2 inflammation characterized by predominant infiltration of T helper-2 T lymphocytes that produce IL-4, IL-5, IL-9 and IL-13. Neutrophils are absent Symptoms in mild to moderate asthmatics are well controlled by treatment with β2-agonists and corticosteroids.

Sarcoidosis

Sarcoidosis and berylliosis are interstitial lung diseases. The interstitium (the space between the tissues) of the lungs includes portions of the connective tissue of the blood vessels and air sacs. Interstitial lung diseases begin with inflammation of the lung cells. The lungs stiffen as a result of inflammation of the air sacs (alveolitis) and scarring (fibrosis) (he Lungs in Health and Disease, National Heart, Lung and Blood Institute; NIH Publication No. 97-3279, August 1997). Sarcoidosis is a systemic granulomatous disease of unknown aetiology and world-wide distribution. It most commonly affects young adults and presents as with bilateral hilar lymphadenopathy, pulmonary infiltration, reticuloendothelial involvement, eye and skin lesions. Shortness of breath (dyspnea) and a cough that will not go away can be among the first symptoms of sarcoidosis (Sarcoidosis, National Heart Lung, and Blood Institute; NIH publication No. 95-3093, reprinted July 1995). The hallmark of pulmonary sarcoidosis is a mononuclear alveolitis which is characterized by activated CD4+lymphocytes, monocytes/macrophages, and non-caseating granulomas. An imbalance in the expression of T1 and T2 cytokines by alveolar cells is thought to play an important role in the immunopathogenesis of sarcoidosis. It is well established that T1 cytokines are important mediators in pulmonary sarcoidosis and that there is a dependence of granulomatous inflammation on T1 cytokines. Alveolar cells spontaneously release the T1 cytokines IFNγ and IL-2 but not T2 cytokines. However, it is suggested that the cytokine patterns change during the course of the disease (Möllers M., et al., 2001). Shigehara K. et al., 2001, demonstrated that IL-12 and IL-18 were increased in BAL fluids of patients with sarcoidosis. IL-12 and IL-18 drive the immune response in to Type 1 direction.

In recent years, new therapies have been studied for sarcoidosis. Drugs used to treat patients with sarcoidosis are corticosteroids, cytotoxic agents, immunomodulators (chloroquine and hydroxychloroquine) and the antileprosy drugs clofazimine and minocycline. A key cytokine in chronic sarcoidosis appears to be TNF. Drugs that inhibit its release or block its bioactivity such as pentoxifylline and thalidomide have been reported to be effective for treatment of sarcoidosis (Baughman R. P., 2002).

Berylliosis

Berylliosis or Chronic Beryllium Disease (CBD) is an environmental chronic inflammatory disorder of the lungs caused by inhalation of insoluble beryllium (Be) dust and characterized by the accumulation of CD4+T cells and macrophages in the lower respiratory tract According to the type and level of Be exposure, the reactions vary from acute tracheobronchitis, chemical pneumonitis, and metal fume fever to a chronic granulomatous lung disorder. Lung T cells respond to beryllium with the release of T1 cytokines such as IL-2, the migration inhibitor factor (MIF, IFNγ, and TNF-α. Furthermore, a positive association with a HLA class II allele, HLA-DP, has been described (Saltini C. et al., 2001). Most patients treated with corticosteroids need to remain on therapy for life (Rossman M. D., 2001).

Cystic Fibrosis

Cystic fibrosis (CF) is an autosomal recessive disorder caused by nearly 1000 different mutations of the cystic fibrosis transmembrane conductance regulator (CFMI) gene. It is a multisystem disorder characterized by defective electrolyte transport in epithelial cells and abnormally viscous mucus secretions from glands and mucus epithelia. Symptoms are pancreatic insufficiency (PI) associated with neonatal meconium ileus and chronic obstructive lung disease superimposed with recurrent opportunistic infections that progressively destroy lung tissue. The inflammatory response is a primary cause of irreversible lung damage. Inflammation is present in CF patients and precedes the bacteria infections, as demonstrated by increased levels of neutrophils,.T and IL-8. Other complications,include liver disease, chronic sinusitis, infertility in male patients and elevated sweat chloride concentrations. Despite advances in genomic technologies and drug discovery, drug therapy often improves disease symptoms but does not cure the disease. One of the main causes of this failure to cure CF may be attributable to genetic variability and to the scarce knowledge of CF biochemistry. The development of new treatments may be important for the life expectancy of patients. Current CF therapeutic strategies include lung transplantation, antimicrobial treatment, corticosteroids, non-steroidal anti-inflammatory drugs like ibuprofen, ion channel therapy, protein-assist therapy, and gene therapy (Sangiuolo F. et al., 2002).

COPD

Chronic obstructive pulmonary disease (COPD) is currently the sixth leading cause of death and the 12th leading cause of morbidity world-wide. By the year 2020, COPD is expected to be the third leading cause of death and the fifth leading cause of disability.

COPD is a disease state characterized by chronic and slow progressive development of airflow limitation that is not fully reversible sad is punctuated by episodic exacerbations due to viral or bacterial infections. The airflow limitation is associated with an abnormal inflammatory response of the lungs to noxious particles or gases (Executive Summary, Global Strategy for the diagnosis, management and prevention of chronic obstructive pulmonary disease; NHLBI/WHO Workshop Report). COPD comprises chronic bronchitis, chronic obstructive bronchiolitis and emphysema (Ockie M. J. et al., 2000). Conic bronchitis is defined as the occurrence of coughing and by production of sputum on most days for at least 3 months over 2 consecutive years. Emphysema is characterized by destructive enlargement of airspaces with loss of normal architecture and lung elasticity.

Cigarette smoking is the dominant factor for the development and progression of COPD. However, only 15% of smokers develop COPD and >15% of COPD-related mortality occurs in people who have never smoked, suggesting that other factors are important Genetic factors such as α₁-antitrypsin deficiency, resulting in enhanced neutrophil elastase activity, account for 2% of emphysema patients and polymorphism of the TNF-αgene, leading to enhanced TNF production, may also play an important role. The role of infections in both the development and progression of COPD is getting increased attention, including adenoviral and rhinoviral infections in patients with emphysema. Occupational and environmental exposures to various pollutants are also considered to be important factors in the development of COPD. (Mannino D. M. et al., 2002; Barnes P. J., 2000).

A diagnosis of COPD should be considered in any patient who has symptoms of cough, sputum production, or dyspnea, and/or a history of exposure to risk factors for the disease inhaled gases (nitric oxide and carbon monoxide) and inflammatory markers in exhaled breath condensate can be used as non-invasive markers of COPD (Leckie M. J. et al., 2000). The diagnosis is confirmed by spirometry. The presence of a post-bronchodilator FEV₁<80% of the predicted value in combination with an FEV₁/FVC <70% confirms the presence of airflow limitation that is not fully reversible (FEV1=forced expiratory volume in one second; FVC =forced vital capacity).

A simple classification of disease severity into four stages is recommended. All FEV₁values refer to post-bronchodilator FEV₁.
Stage 0: At Risk: characterized by chronic cough and sputum production. Lung function, as measured by spirometry, is still normal.
Stage I: Mild COPD: characterized by mild airflow limitation (FBV₁/FVC<70% but FEV₁80% predicted) and usually, but not always, by chronic cough and sputum production.
Stage II: Moderate COPD: characterized by worsening airflow limitation (30% ≦FEV₁<80% predicted) and usually the progression of symptoms, with shortness of breath typically developing on exertion This is the stage at which patients typically seek medical attention because of dyspnea or an exacerbation of their disease. The division into stages IIA and IIB is based on the fact that exacerbations are eely seen in patients with an FEV₁ below 50% predicted The presence of repeated exacerbations has an impact on the quality of life of patients and requires appropriate management
Stage III: Severe COPD: characterized by severe airflow limitation (FEV₁<30% predicted) or the presence of respiratory failure or clinical signs of right heart failure. Patients may have severe (Stage III) COPD even if the FEV₁ is >30% predicted, whenever these complications are present. At this stage, quality of life is appreciably impaired and exacerbations may be life-threatening.

COPD is generally regarded as a separate condition from asthma (reversible airflow limitation) in terms of inflammatory processes, underlying pathology, and responses to treatment Airway inflammation in asthma is characterized by infiltration of eosinophils and T helper-2 lymphocytes. Macrophages are less frequent and CD8 T cells are usually absent The T2 cytokines predominate: IL-4 and IL-13 play an important role in IgE production, whereas IL-5 is critical for eosinophil growth and differentiation (Barnes P. J., 2000).

Airway inflammation in COPD is characterized by the presence of neutrophils, macrophages and CD8 T cells. Histopathological studies show that most inflammation in COPD occurs in the peripheral airways (bronchioles) and lung parenchym. The bronchioles are obstructed by fibrosis and there is destruction of lung parenchym. Bronchial biopsy results show similar find There is also a marked increase in macrophages and neutrophils observable in bronchoalveolar lavage fluid and induced sputum.

Emphysema is caused by an imbalance of proteases and protease inhibitors. The concentration of inflammatory mediators such as leukotriene B₄, TNF-α and IL-8 are increased in sputum of patients with COPD (Barnes P. J., 2000). Other inflammatory cytokines associated with COPD are TGF-β, IL-1, IL-6, IL-11 and IL-18. Also a role for IFNγ in COPD has been mentioned in the literature, however with some differing data Wang Z. et al. (2000) describe a transgenic mouse model, with IFNγ inducibly targeted to the adult murine lung, showing emphysema, macrophage and neutrophil infiltration and inversed proteasetanti-protease ratio. Majori M. et al.(1999) showed an increase in the percentage of IFNγ-producing cells among peripheral blood T helper cells from patients with COPD. Analysis of cytokine levels in BAL (bronchoalveolar lavage) discloses a prevalent T1 cytokine pattern in COPD, however this difference was not significant when COPD patients were compared with the other two groups (asthmatics and non-smoker) (Balbi B. et al., ATS 2002). Lethbridge M. W. G. et al. (2002) even showed a disproportionately low number of IFNγ-expressing airway lymphocytes in COPD smokers as compared to healthy smokers and healthy ex-smokers. The association of chronic inflammation with the pathophysiology of COPD makes IL-1, IL-18, and TNF-αtargets for therapeutic intervention (de Boer W. L., 2002). Macrophages predominate and appear to play a central role as they have the capacity to produce all the pathologic changes of COPD (Hautamaki, 1997). Macrophages release LTB4 and 1L-8, which are potent neutrophil chemo-attractants, and multiple proteases responsible for the continued proteolytic activity in the lungs of patients with emphysema.

At the present time, there are no known drugs that slow the relentless progression of COPD and there is a pressing need to develop new drugs to control the inflammatory and destructive processes that underlie the disease. Smoking cessation is the only measure that will slow the progression of COPD. However, even if the patient stops smoking, the damage already caused will continue to cause symptoms (Banes P. J., 2001). Currently available drugs that provide symptomatic relief in COPD are:
Bronchodilators, which are the mainstay of current drug therapy for COPD (Leckie M. J. et al., 2000).
Inhaled corticosteroids are also widely prescribed for COPD but have a risk of systemic side effects (Barnes P. J., 2000). The usefulness of inhaled corticosteroids in COPD remains controversial (Leckie M. J. et al., 2000).
Theophylline
Although antibiotics are still widely used for exacerbations of COPD, it is increasingly recognized that exacerbations may be due to vial infections of the upper respiratory tract or may be noninfective, so that antibiotic treatment is not always warranted (Barnes P. J., 2002).

Nonpharmarcologic treatments include oxygen therapy, non-invasive ventilation, exercise training, pulmonary rehabilitation and lung volume reduction surgery (Barnes P. J., 2000).

A better understanding of the cellular and molecular mechanisms involved in COPD provides new molecular targets for the development of drugs and several classes of new drugs are now under development Leukotriene B₄ (LT B₄) inhibitors, chemokine inhibitors, TNF-α inhibitors, antioxidants, iNOS inhibitors, and corticosteroids are examples of inflammatory mediator antagonists. Examples of protease inhibitors are neutrophil elastase inhibitors, cathepsin, inhibitors, α₁-antitrypsin, secretory leukoprotease inhibitor and elafin New anti-inflammatory drugs for COPD are PDE type IV inhibitors, NF-κB inhibitors, adhesion molecule blockers, IL-10, p38 MAP kinase inhibitors, and P13-kinase inhibitors (Banes P. J., 2001).

As described earlier, currently available therapies for T1 inflammatory lung disease are broad acting and directed to the improvement of clinical symptoms and/or to a general reduction of inflammation. There is thus a need for new selective therapeutic strategies which end the relentless progression of said diseases by targeting a key mediator of the underlying mechanism.

Notwithstanding the fact that several potential therapies for T1 mediated inflammatory lung disease have been proposed, no prior art exists revealing that neutralizing IFNγ bioactivity is effective in the treatment of T1 mediated inflammatory lung diseases such as COPD, severe asthma, sarcoidosis, berylliosis, and cystic fibrosis.

The present invention demonstrates that the inflammatory and destructive processes that underlie T1 inflammatory lung diseases can be treated by neutralizing IFNγ.

### SUMMARY OF THE INVENTION

It is an object of the present invention to provide methods and compositions for preventing or treating T1 inflammatory lung disease, particularly COPD, emphysema, chronic bronchitis, bronchiolitis, severe asthma, sarcoidosis, berylliosis, and cystic fibrosis.

The present invention provides a method for preventing or treating T1 inflammatory lung disease, said method comprising reducing or neutralizing the bioactivity of IFNγ. Several methods and compositions can be applied for this and are thus part of the current invention.

An aspect of the present invention relates to the use of an IFNγ neutralizing molecule for preventing or treating T1 inflammatory lung disease. More particularly, the present invention relates to the use of an anti-IFNγ antibody for preventing or treating T1 inflammatory lung disease, said antibody preferably being a monoclonal antibody. Furthermore, the present invention relates to the use of a human or a humanized anti-IFNγ antibody for preventing or treating T1 inflammatory lung disease. More specifically, the present invention relates to the use of the anti-IFNγ antibody D9D10, and more particularly a humanized ant-IFNγ antibody D9D10, for preventing or treating T1 inflammatory lung disease.

Another aspect of the present invention relates to the use of immunogenic IFNγ for preventing or treating T1 inflammatory lung disease, and in particular the use of human immunogenic IFNγ.

Accordingly, the present invention also relates to the prevention or treatment of T1 inflammatory lung disease by immunization with a pharmaceutical composition comprising immunogenic IFNγ proteins and/or IFNγ derived (poly)peptides. Several techniques to render IFNγ immunogenic are well known in the art and the present invention allows for all kinds of permutations of the original IFNγ sequence, and all kinds of modifications therein.

Another aspect of the invention relates to the use of the technology of genetic immunization, also known as “DNA vaccination”, for preventing or treating T1 inflammatory lung disease.

### DETAILED DESCRIPTION OF THE INVENTION

Unless defined otherwise, all technical and scientific terms used herein have the same meaning as commonly understood by one of ordinary skill in the art. Although any methods and materials similar or equivalent to those described herein can be used in the practice or testing of the present invention, the preferred methods and materials are now described. All publications mentioned hereunder are incorporated by reference. Unless mentioned otherwise, the techniques employed herein are standard methodologies well known to one of ordinary skill in the art. The materials, methods and examples are only illustrative not limiting.

The present invention provides compositions and methods for preventing and treating T1 inflammatory lung disease. Said lung diseases include but are not limited to COPD (comprising emphysema, chronic bronchitis, and bronchiolitis), severe asthma, sarcoidosis, berylliosis and cystic fibrosis. The methods of the invention encompass the neutralization and/or reduction and/or blockade of IFNγ bioactivity. The current invention thus relates to a method for preventing or treating T1 inflammatory lung disease, said method comprising the neutralization of IFNγ bioactivity. Several methods and/or compositions can be used in order to achieve said effect and will be described hereunder.

Administration of IFNγ Neutralizing Molecules.

A first aspect of the invention is directed to the use of a molecule capable of neutralizing and/or reducing and/or fully inhibiting the bioactivity of IFNγ for preventing or treating T1 inflammatory lung disease. More specifically, the invention relates to the use of an IFNγ neutralizing molecule for the manufacture of a medicament for preventing or treating a T1 inflammatory lung disease.

A “T1 inflammatory lung disease” is characterized by a Type 1 immune response mediated by T helper-1 cells (CD4+) and T cytotoxic-1 cells (CD8+) and by predominant production of interferon gamma (IFNγ), tumor necrosis factor (IFNγ) and interleukin-2 (IL-2). T1 cytokines evoke cell-mediated immunity characterized by prominent lung tissue infiltration of macrophages, neutrophils and T-cells. Examples of T1 inflammatory lung diseases include, but are not limited to COPD, emphysema, chronic bronchitis, bronchiolitis, severe asthma sarcoidosis, berylliosis, and cystic fibrosis.

As used herein, the term “molecule” encompasses, but is not limited to, an antibody and fragments thereof, a diabody, a triabody, a tetravalent antibody, a peptide, a low molecular weight non-peptide molecule (also referred to as “small molecules”) and a (soluble) IFNγ receptor or fragments thereof, which specifically reduces and/or inhibits IFNγ bioactivity. IFNγ of any species, including humans, is to be considered in this respect.

As used herein, the term “antibody” refers to monoclonal antibodies, polyclonal antibodies, antibodies which are derived from a phage library, humanized antibodies, synthetic antibodies, chimeric antibodies, antibody fragments, single-chain Fv's, or constructs thereof. The term “monoclonal antibody” refers to an antibody composition having a homogeneous antibody population. The term is not intended to be limited by the manner in which it is made. A monoclonal antibody typically displays a single binding affinity for a particular polypeptide with which it immunoreacts. A monoclonal antibody to an epitope of the IFNγ antigen can be prepared by using a technique which provides for the production of antibody molecules by continuous cell lines in culture. These include but are not limited to the hybridoma technique originally described by Kohler and Milstein (1975). Monoclonal antibodies can also be produced in various ways using techniques well understood by those having ordinary skill in the art. Details of these techniques are described in “Antibodies: A Laboratory Manual”, Harlow et al.(ed), Cold Spring Harbor Publications, p. 726 (1988), or are described by Campbell, A. M. (“Monoclonal Antibody Technology Techniques in Biochemistry and Molecular Biology,” Elsevier Science Publishers, Amsterdam, The Netherlands (1984)) or by St Groth et al.( J. Immunol. Methods 35:1-21 (1980)). Monoclonal antibodies of any species, including humans, can be used in this invention. Accordingly, the antibodies according to this embodiment may be human monoclonal antibodies. Such human monoclonal antibodies may be prepared, for instance, by the generation of hybridomas, derived from immunised transgenic animals, containing large sections of the human immunoglobulin (Ig) gene loci in the germline, integrated by the yeast artificial chromosomal (YAC) technology (Mendez et al., 1997). Also fragments derived from these monoclonal antibodies such as Fab, F(ab)′₂ and scFv (“single chain variable fragment”), providing they have retained the original binding properties, form part of the present invention. The present invention thus also relates to the use of an anti-IFNγ antibody for the manufacture of a medicament for preventing or treating T1 inflammatory lung disease, wherein said antibody is a monoclonal or polyclonal antibody, and more particularly a human monoclonal or polyclonal antibody.

As used herein, the term “humanized antibody” means that at least a portion of the framework regions of an immunoglobulin or engineered antibody construct is derived from human immunoglobulin sequences. It should be clear that any method to humanize antibodies or antibody constructs, as for example by variable domain resurfacing as described by Rogusim et al. (1994) or CDR grafting or reshaping as reviewed by Hurle and Gross (1994), can be used.

As used herein, the term “chimeric antibody” refers to an engineered antibody construct comprising variable domains of one species (such as mouse, rat, goat, sheep, cow, llama, or camel variable domains), which may be humanized or not, and constant domains of another species (such as primate or human constant domains) (for review see Hurle and Gross (1994)). It should be clear that any method known in the art to develop chimeric antibodies or antibody constructs can be used.

As used herein, the term “single chain Fv”, also termed scFv, refers to engineered antibodies prepared by isolating the binding domains (both heavy and light chains) of a binding antibody, and supplying a linking moiety which permits preservation of the binding function. This forms, in essence, a radically abbreviated antibody, having only that part of the variable domain necessary for binding the antigen. Information concerning the generation, design and expression of recombinant antibodies can be found in Mayforth RD, “Designing Antibodies”, Academic Press, San Diego (1993).

As used herein, the term “fragment” or “fragments” refers to F(ab), F(ab)′2, Fv, scFv and other fragments which retain the antigen binding function and specificity of the parent antibody. The methods for producing said fragments are well known to a person skilled in the art and can be found, for example, in Antibody Engineering, Oxford University Press, Oxford (1995) (1996) and Methods in Molecular Biology, Humana Press, New Jersey (1995). In addition, any construct of an antibody or a fragment is also a subject of the current invention. As used herein, the term “construct” relates to synthetic or recombinant molecules, including but not limited to diabodies, triabodies, tetravalent antibodies, pepta- or hexabodies, and the like, that are derived from an anti-IFNγ antibody. The present invention thus relates to the use of an IFNγ neutralizing molecule for the manufacture of a medicament for preventing or treating a T1 inflammatory lung disease, whereby said molecule is a construct derived from an anti-IFNγ antibody.

As used herein, the term “diabody” relates to two non-covalently-linked scFv's, which then forming a so-called diabody, as described in detail by Holliger et al. (1993) and reviewed by Poljak (1994). It should be clear, that any method to generate diabodies, as for example described by Holliger et al. (1993), Poljak (1994), and Zhu et al. (1996), can be used.

As used herein, the term “triabody” relates to trivalent constructs comprising 3 scFv's, and thus comprising 3 variable domains, as described by Kortt et al. (1997) and Iliades et al. (1997). A method to generate triabodies is described by Kortt et al. (1997). An example of a triabody is given in WO 99/09055 by Innogenetics N.V. It should also be clear that the scFv's, chimeric antibodies, diabodies and triabodies described above are not limited to comprise the variable domain of the same antibody but may also comprise variable domains of other anti-IFNγ antibodies which efficiently reduce or neutralize the bioactivity of IFNγ. Furthermore, the diabodies or triabodies described above may also comprise two scFv's of different specificities. For example, the latter diabodies may simultaneously neutralize IFNγ on the one hand and may target another molecule, such as TNF-α, IL-1, IL-2, B7.1 or CD80, B7.2 or CD86, IL-12, IL-4, IL-10, CD40, CD40L, IL-6, complement factor, coagulation factor, fibrinolysis factor, tumour growth factor-beta (TGF-β), transferin receptor, insulin receptor and prostaglandin E2, or any other molecule, on the other hand.

As used herein the terms “IFNγ neutralizing molecule” or “IFNγ neutralizing antibody” refer to a molecule and an antibody which inhibits or blocks any bioactivity of IFNγ, respectively.

The term “bioactivity” or “biological activity” of IFNγ relates to the antiviral activity (Billiau, 1996), the induction of the expression of MHC-class-II molecules by macrophages and other cell types (Steinman et al., 1980), the stimulation of the production of inflammatory mediators such as TNF-α, IL-1 and NO (Lorsbach et al., 1993), the induction of the expression of adhesion molecules such as ICAM-1 (Dustin et al., 1988) and of important costimulators such as the B7 molecules on professional antigen presenting cells (Freedman et al., 1991), the induction of macrophages to become tumoricidal (Pace et al., 1983), the induction of Ig isotype switching (Snapper and Paul, 1987) or any other known bioactivity of IFNγ. Billiau et al. (1996) describes pathological and/or clinical activity during diseases in which IFNγ is pathogenic It should be noted that the molecules which neutralize IFNγ as described herein, neutralize at least one bioactivity but not necessarily all bioactivities of IFNγ. Tests to evaluate the effect of anti-IFNγ molecules or antibodies (i.e. IFNγ, neutralizing molecules or antibodies resp.).on the bioactivity of IFNγ are available and well known to the skilled person. Examples of said tests are, but not limited to, “inhibition of MHCII-induction” and/or “inhibition of anti-viral activity”. In the first mentioned test, the effect of IFNγ on the induction of M19C class II expression on keratinocytes is examined. For this, primary keratinocytes are cultured with two concentrations of IFN-γ (100 U/ml and 200 U/ml) for 24 and 48 hours. After culture, cells are collected and the expression of MHC class II antigen on the activated keratinocytes is measured by FACS-scan after staining (30 minutes at 4° C) of the cells with a PE-labelled anti-MHC-class II mAb. In addition, the effect of an anti-IFNγmolecule on the IFNγ-induced MHC-Class II expression on keratinocytes is examined. In this experiment, primary keratinocytes are cultured with IFNγ (100 U/ml) in the presence or absence of different concentrations of anti-IFNγ molecules or antibodies for 48 hours. IFNγ is preincubated with anti-IFNγ molecules or antibodies for 1 hour at 37° C. before adding to the keratinocytes. After culture, cells are collected and the expression of MHC-Class II on these activated keratinocytes is measured. For this, keratinocytes are incubated (30 minutes at 4° C.) with a PE-labelled anti-MHC-ClassII mAb (Becton Dickinson), washed twice with PBS and fixed. The MHC-Class ll expression is further analysed on a FACS-scan. Analogous to the described test, the effect of IFNγ on the induction of MHC-class II expression on B cells can be examined. Also other experiments known to those skilled in the art can be performed in order to evaluate the neutralization capacity of anti-IFNγ molecules (incl. antibodies).

For the second test, whereby neutralization of the antiviral activity of IFNγ is measured, serial dilutions of samples (anti-IFNγ molecules or antibodies) are prepared in microtiter plates. IFNγ is added to each well in a final concentration of 5 antiviral protection Units/ml, as tested on A549 cells. The mixtures are incubated for 4 h at 37° C. and 25000 A549 cells are added to each well. After an incubation period of 24 at 37° C. a in CO₂ incubator, 25 μl of 8×10⁵PFU EMC virus/ml is added to the cultures for at least 24 h. As soon as virus-infected control cultures reach 100% cell destruction, a crystal violet staining is performed in order to quantify surviving cells. The neutralization capacity of the anti-IFNγ molecules or antibodies can be defined for instance, as the concentration of the molecule or antibody needed to neutralize 95% of the antiviral activity of 5U/ml IFNγ. The neutralization potency of the anti-IFNγ molecules or antibodies is then determined. Further assays to evaluate the effect of anti-IFNγ molecules (incl. antibodies) on the bioactivity of IFNγ are described by e.g Lewis J. A. (1995), Kim Y. & Son K (1996) and by Maeger A. (2002).

The term “prevention” or “treatment” as used herein refers to either (i) the prevention of the disease of interest (prophylaxis), or (ii) the reduction or elimination of symptom exacerbations or the disease of interest (therapy), or (iii) any process, action, application, therapy, or the like, wherein a mammal, including a primate and more specifically a human being, is subject to medical aid with the object of improving the mammal's condition, directly or indirectly.

The present invention thus relates to a method for preventing or treating T1 inflammatory lung disease comprising administering to a patient a pharmaceutically effective amount of an IFNγ neutralizing molecule. More specifically, the present invention relates to a method for preventing or treating T1 inflammatory lung disease comprising administering to a patient a pharmaceutically effective amount of an anti-IFNγ antibody. According to a specific embodiment, the antibody is the monoclonal antibody D9D10H3G5 produced by the hybridoma deposited on Aug. 28, 2001, under the Accession No. DSM ACC2521, with the DSMZ-Deutsche Sammlung von Mikroorganismen und Zellkulturen GmbH, Mascheroder Weg 1b, D-38124 Braunschweig, Germany. Said monoclonal antibody D9D10H3G5 will be further abbreviated throughout the specification and the claims as D9D10. More specifically, the present invention relates to a method for preventing or treating T1 inflammatory lung disease comprising administering to a patient a pharmaceutically effective amount of an anti-IFNγ antibody D9D10 or a fragment thereof. Furthermore, the present invention relates to the use of an anti-IFNγ antibody for the manufacture of a medicament for preventing or treating T1 inflammatory lung disease, said antibody preferably being a monoclonal antibody (e.g. D9D10 as described herein) or a humanized monoclonal antibody (e.g. Humanized D9D10 as described herein or as described in W099109055 by Innogenetics N.V.).

Differently produced antibodies recognizing the same epitopes as the antibody D9D10, as well as antibodies immunologically competing with the antibody D9D10 for the binding on IFNγ are also part of the invention. Therefore, according to a further embodiment, the present invention relates to the use of an anti-IFNγ antibody or a fragment thereof for preventing or treating T1 inflammatory lung disease, whereby said antibody is characterized by its ability to immunologically compete with the antibody D9D10 for the binding on IFNγ. As used herein, the term “to bind in an equivalent way” or “immunologically competing” means that these antibodies inhibit the binding of D9D10 to IFNγ and that these antibodies neutralize the bioactivity of IFNγ. Preferred methods for determining antibody specificity and affinity by competitive inhibition, e.g. solid phase ELISA, can be found in Harlow et al. (1988), Colligan et al. (1992, 1993), Ausubel et al. (1987, 1992, 1993), and Muller R. (1993), and in Karlsson et al. (1991) and Malmqvist M. (1999).

As used herein, the tern “pharmaceutical composition” or “composition” or “medicament” refers to any composition comprising a molecule, including an antibody or fragment thereof, which specifically neutralizes IFNγ, preferably in the presence of a pharmaceutically acceptable carrier or excipient. The present invention thus also relates to the use of a pharmaceutical composition comprising at least one IFNγ neutralizing molecule and an acceptable carrier for preventing or treating T1 inflammatory lung disease. More preferably, said composition comprises the antibody D9D10 or a humanized D9D10 antibody and a pharmaceutically acceptable carrier. Further, said composition optionally comprises other drugs or other antibodies, antibody derivatives or constructs. With regard to COPD, emphysema, chronic bronchitis and chronic obstructive bronchiolitis, examples of such other drugs or other antibodies, antibody derivatives or constructs are, but are not limited to: bronchodilators, corticosteroids, Theophylline, antibiotics, Leukotriene B₄ (LT B₄) inhibitors, chemokine inhibitors, TNF-α inhibitors, anti-IL-8, antioxidants, iNOS inhibitors, neutrophil elastase inhibitors, cathepsin inhibitors, α₁-antitrypsin, secretory leukoprotease inhibitors, elafin, PDE type IV inhibitors, NF-κB inhibitors, adhesion molecule blockers, IL-10, p38 MAP kinase inhibitors, P13-kinase inhibitors and TNF-tip peptides as described in WO 00/09149; with regard to severe asthma: β2-agonists, anticholinergics, corticosteroids (e.g. prednisone); with regard to sarcoidosis: corticosteroids, cytotoxic agents, immunomodulators (e.g. chloroquine, hydroxychloroquine and TNF inhibitors such as pentoxifylline and thalidomide) and the antileprosy drugs clofazimine and minocycline; with regard to berylliosis: corticosteroids; with regard to cystic fibrosis: antimicrobial agents, corticosteroids and non-steroidal anti-inflammatory drugs such as ibuprofen.

It should also be clear that any possible mixture of any IFNγ neutralizing molecule, antibody or composition described in the specification may be part of the above-indicated pharmaceutical composition. The proportion and nature of said pharmaceutical compositions are determined by the solubility and chemical properties of the selected compound, the chosen route of administration, and standard pharmaceutical practice.

The IFNγ neutralizing molecule, antibody or a fragment thereof, and more preferred the monoclonal antibody D9D10 or a humanized D9D10 antibody, or a fragment or construct thereof, may thus be administered or delivered in the form of any suitable composition as described in the specification by any suitable method of administration within the knowledge of one skilled in the art.

As used herein, the term “pharmaceutically acceptable carrier or excipient”, whereby the term carrier and excipient are used interchangeably, refers to a diluent, adjuvant, or vehicle with which the therapeutic molecule is administered. It includes any and all solvents, dispersion media, aqueous solutions, coatings, antibacterial and antifungal agents, isotonic and absorption delaying agents, and the like. The use of such media and agents for pharmaceutically active substances is well known in the art. Except insofar as any conventional media or agent is incompatible with the active ingredient, the use thereof in pharmaceutical compositions is contemplated. Supplementary active ingredients can also be incorporated into the compositions of the invention A composition is said to be “pharmacologically acceptable” if its administration can be tolerated by the recipient.

Immunization.

Another method to neutralize the bioactivity of IFNγ is by immunization. There is an increased focus on methods of instructing the recipient's own immune system to generate endogenous antibodies of the appropriate specificity by means of immunization. However, mammals do not generally have high-titre antibodies against self-proteins in serum because of homeostatic tolerance mechanisms that prevent their formation (autotolerance).

It has been shown (by Dalum I. et al., 1996) that potentially self-reactive B lymphocytes recognizing self-proteins are physiologically present in normal individuals. However, in order for these B lymphocytes to be induced to actually produce antibodies reactive with the relevant self-proteins, assistance is needed from cytokine producing T helper lymphocytes (Th-cells or Th-lymphocytes). Normally this help is not provided because T lymphocytes in general do not recognize T-cell epitopes derived from self-proteins when presented by antigen presenting cells (APCs) such as dendritic cells, macrophages and B cells. However, by providing an element of “foreignness” in a self-protein, T-cells recognizing the foreign element are activated upon recognizing the foreign epitope on an APC (such as, initially, a mononuclear cell). Polyclonal B lymphocytes (which are also specialised APCs) capable of recognizing self-epitopes on the modified self-protein, also internalize the antigen and subsequently present the foreign T-cell epitope(s) thereof, and the activated T lymphocytes subsequently provide cytokine help to these self-reactive polygonal B lymphocytes. Since the antibodies produced by these polyclonal B lymphocytes are reactive with different epitopes on the modified polypeptide, including those which are also present in the native polypeptide, an antibody cross-reactive with the non-modified self-protein is induce. In conclusion, the T lymphocytes can be led to act as if the population of polyclonal B lymphocytes have recognized an entirely foreign antigen, whereas in fact only the added or inserted epitope(s) is/are foreign to the host. In this way, antibodies capable of cross-renting with non-modified self-antigens are induced.

Different kinds of immunization approaches that are able to break B-cell tolerance and circumvent antibody tolerance mechanisms without inducing auto-antibody-mediated pathology and toxicology are well known by the skilled person and are outlined hereunder.

Accordingly, the present invention relates to the prevention or treatment of T1 inflammatory lung disease by immunization with a pharmaceutical composition comprising immunogenic IFNγ proteins and/or IFNγ derived (poly)peptides. These IFNγ-related compounds are capable of raising auto-antibodies and/or T cells when administered in vivo. The autoantibodies are able to neutralize and inhibit the biological activities of endogenously produced IFNγ. The present invention thus relates to the use of immunogenic IFNγ or IFNγ derived (poly)peptides for the manufacture of a medicament for preventing or treating a T1 inflammatory lung disease.

“Immunization” means that a substance or composition of matter exhibits or induces an immune response resulting in endogenous antibody production concomitant with or without T cell help. Endogeneous antibody production against IFNγ can be measured using standard techniques, e.g. by ELISA.

The term “immunogen” is intended to denote a substance which is capable of inducing an immune response in a certain mammals, including primates and more specifically humans. It will therefore be understood that autologous IFNγ is not an immunogen in the autologous host under non-pathogenic conditions. It is necessary to use either a strong adjuvant and/or to co-present foreign T helper epitopes with the autologous INFγ in order to mount an immune response against autologous IFNγ and in such a case the “immunogen” is the composition of matter which is capable of breaking autotolerance. Other immunogens described in the current invention include but are not limited to modified IFNγ, non-self IFNγ (i.e. IFNγ from another species) and antigen presenting cells loaded with IFNγ. The term “immunogenic IFNγ” thus specifically relates to:
modified IFNγ and/or,
non-self IFNγ derived from another species (mammalian or other) administered with or without an adjuvant and/or,
autologous IFNγ co-presented or in combination with an adjuvant and/or,
autologous antigen presenting cells, such as dendritic cells, loaded with IFNγ.

The expressions “IFNγ”, “IFNγ protein” and “IFNγ polypeptide”, which are used interchangeably, refer to a family of polypeptide molecules that include human IFNγ from natural sources, synthetically produced in vitro, or obtained by genetic manipulation including methods of recombinant DNA technology. The amino acid sequence variants preferably share at least about 65% sequence homology, more preferably at least about 75% s c homology, even more preferably at least about 85% sequence homology, most preferably at least abut 90% sequence homology with any domain, and preferably with the receptor binding domain(s) of the native human IFNγ amino acid sequence. Several databases and tools for determining amino acid homology are known by the person skilled in the art, e.g. BLAST®, and are described by Gish W. & States D. J. (1993) and in “Bioinformatics: sequence and genome analysis”, Mount (ed.), Cold Spring Harbor Laboratory Press (2001). The definition specifically covers variously glycosylated and unglycosylated forms of native human IFNγ and of its amino acid sequence variants.

A “modified IFNγ” is an protein or IFNγ derived (poly)peptide which has been subjected to changes in its primary structure. Such a change can e.g. be in the form of fusion or conjugation of an IFNγ polypeptide to a suitable fusion partner (i.e. a change in primary structure exclusively involving C-and/or N-terminal additions of amino acid residues) and/or it can be in the form of insertions and/or deletions and/or substitutions in the amino acid sequence of IFNγ. It should also be noted that the IFNγ protein can become immunogenic due to certain modifications resulting from the recombinant production process, isolation, handling, or storage of the protein. According to a preferred embodiment of the present invention the IFNγ protein is rendered immunogenic by its modification.

One technique involves chemically crosslinking of the IFNγ self-protein (or (poly)peptide(s) derived from it) to a highly immunogenic non-self carrier protein such as, but not limited to, keyhole limpet haemoncyanin (Kim, 2001), ovalbumin (Richard, 2000), tetanus toxoid, detoxified difteria or cholera toxin (Talwar, 1999), bacterial outer membrane proteins (Gonzalez, 2000), E.coli enterotoxin (Lowenadler, 1994), heat shock proteins (Udono, 1993), and viral-like particles (Chackerian, 2002; Storni, 2002). The carrier protein contains several different foreign T-cell epitopes needed to trigger the activation of T-cells which in their turn provide help to B-cells that produce antibodies specifically directed against the conjugated IFNγ self-antigen. Instead of whole carrier proteins, the chemical cross-linking of foreign MHC Class II restricted T-cell epitopes may also be efficient for the induction of auto-immune responses against IFNγ (Sad, 1992). The current invention thus relates to the use of immunogenic IFNγ for the manufacture, of a medicament for preventing or treating a T1 inflammatory lung disease, whereby the IFNγ protein is modified to immunogenic IFNγ by crosslinking IFNγ to an immunogenic non-pelf carrier protein. Furthermore, the current invention also relates to the use of immunogenic IFNγ for the manufacturing of a medicament for preventing or treating a T1 inflammatory lung disease, whereby the IFNγ protein is modified to immunogenic IFNγ by chemical cross-linking of one or more foreign MHC Class II restricted T-cell epitopes.

A variant on the carrier protein technique involves the construction of a gene encoding a fusion protein comprising both carrier protein or foreign T-cell epitope(s) and the IFNγ self-protein or B-cell epitope(s) derived therefrom. The fusion protein may be pressed in a suitable host cell in vitro, the gene product purified and then delivered as a conventional pharmaceutical composition co-presented with or without adjuvant. The current invention thus also relates to the use of a fusion protein for the manufacture of a medicament for preventing or treating a T1 inflammatory lung disease, whereby the fusion protein comprises a carrier protein or at least one foreign T-cell epitope and the IFNγ self-protein or B-cell epitope(s) derived therefrom. Alternatively, the fusion gene may be administered directly as part of a nucleic acid vaccine.

The foreign T-cell epitope(s) can not only be conjugated to the amino- or carboxy-terminal end of the human IFNγ self-protein but can alternatively be inserted into the human IFNγ protein either at random or at sites predicted not to interfere with the general conformational structure. As a result, T-cell help arises either from this epitope(s) or from junctional sequences.

A more refined approach has been described by Dalum and colleagues wherein a small part of target molecule is substituted by a single MHC Class II restricted T cell epitope (Dalum I., 1999). The same approach can be used to introduce multiple epitopes by substitution. The substituted epitope is supposed not to interfere with the folding of the protein resulting in the adoption of a fully native conformation identical to the one found in the native endogenous self-protein Therefore, it is highly probable that antibodies recognizing the immunogen will also recognize and at best neutralize the endogenous self-protein.

One preferred version of this embodiment is the technique described in WO 95/05849 by Elsner Henrik et al., which discloses a method for down-regulating self-proteins by immunizing with analogues of the self-proteins wherein a number of amino acid sequence(s) has been substituted with a corresponding number of amino acid sequence(s) which each comprise a foreign immunodominant T-cell epitope, while at the same time maintaining the overall tertiary structure of the self-protein in the analogue. For the purposes of the present invention it is, however, sufficient if the modification (be it an amino acid insertion, addition, deletion or substitution) gives rise to a foreign T-cell epitope and at the same time preserves a substantial number of the B-cell epitopes in IFNγ. The present invention thus relates to the use of immunogenic IFNγ for the manufacture of a medicament for preventing or treating a T1 inflammatory lung disease, wherein the IFNγ protein is modified to immunogenic IFNγ by introducing at least one foreign T-cell epitope into IFNγ by insertion, substitution, addition and/or conjugation. Said foreign T-cell epitope can be any natural or synthetic T-cell epitope as described herein.

A “foreign T-cell epitope” is a peptide which is able to bind to an MHC molecule and stimulates T-cells in an animal species. Preferred foreign epitopes are “promiscuous” epitopes, i.e. epitopes which bind to a substantial fraction of polymorphic MHC class II molecules in an animal species. A term which is often used interchangeably in the art is the term “universal T-cell epitopes” for this kind of epitope.

A number of naturally occuring “promiscuous” T-cell epitopes exist which are active in a large proportion of individuals of an animal species, including human, and these are preferably introduced in the IFNγ composition thereby reducing the need for a very large number of different analogues in the same composition.

The promiscuous epitope can according to the invention be a naturally occurring human T-cell epitope such as epitopes from tetanus toxoid (e. g. the P2 and P30 epitopes in WO 00/20027), diphtheria toxoid, influenza virus hemagluttinin (HA), and P. falciparum CS antigen.

Over the years a number of other promiscuous T-cell epitopes have been identified. in particular, peptides capable of binding a large proportion of HLA-DR molecules encoded by the different HLA-DR alleles have been identified and these are all possible T-cell epitopes to be introduced in analogues used according to the present invention. Additional epitopes are discussed in the following references: Kilgus et al., 1991; Contreras et al., 1998; Doolan et al., 2000, Launois et al., 1994; Mustafa et al., 2000; Fernando et al., 1995; Gaudebout et al., 1997; Friedl-Hayek et al., 1999; Kobayashi et al., 2000. All epitopes listed in these references are relevant as candidate epitopes to be used in the present invention, as are epitopes which share common motifs with these.

Alternatively, the epitope can be any synthetic or artificial T-cell epitope which is capable of binding a large proportion of haplotypes. In this context the pan DR epitope peptides (“PADRE”) described in WO 95/07707 and in the corresponding paper by Alexander J. et al. (1994) are interesting candidates for epitopes to be used according to the present invention.

Another mechanism provides for the generation of a multiplicity of potential T-cell epitopes, yet simultaneously retains the target molecule in a conformation close to the native form (Ciapponi,1997). These properties can be achieved by rendering one or several mutations in a self-protein to produce a sequence at those points which can be found in an analogous protein from a second mammalian species. Mutations can be applied to consecutive amino acids or locally dispersed amino acids.

The current invention relates to the use of immunogenic IFNγ for the manufacture of a medicament for preventing or treating a T1 inflammatory lung disease, whereby the IFNγ protein is modified to immunogenic IFNγ by rendering one or several mutations in the IFNγ self-protein. Such mutations may in effect produce a sequence at those points which can be found in an analogous protein from a second mammalian species. Alternatively, B-cell epitopes of IFNγ from a first ma an species may be grafted (e.g. by insertion or substitution) into the framework of a protein from a second mammalian species such that the modified protein is able to raise in the first species an immunogenic response directed to the natural IFNγ protein from which the B-cell epitopes are derived. A specific embodiment of the current invention thus relates to the use of immunogenic IFNγ for the manufacture of a medicament for preventing or treating a T1 inflammatory lung disease, whereby a protein from a second mammalian species is modified to an immunogen by B-cell epitopes from an IFNγ protein from a first mammalian species into the frame work of protein from the second mammalian species. Preferably, the first mammalian species is human.

Auto-antibodies in humans can also be induced by immunization with a non-modified analogous protein from a second mammalian species (Vernersson, 2002). Antibodies directed against the analogous protein are suspected to cross-react with the human self-protein resulting in the neutralization of its biological activity. The invention thus also relates to the use of immunogenic IFNγ for the manufacture of a medicament for preventing or treating a T1 inflammatory lung disease, whereby the immunogenic IFNγ is a non-self IFNγ derived from a second mammalian species.

Anti-human IFNγ auto-antibodies in humans can also be achieved by immunization with autologous antigen presenting cells (APCs) ex vivo loaded with human IFNγ (Li, 2002). The invention thus also relates to the use of at least one autologous antigen presenting cell loaded with IFNγ for the manufacture of a medicament for preventing or treating a T1 inflammatory lung disease. More particularly, the invention relates to the use of at least one autologous dendritic cell loaded with IFNγ for the manufacture of a medicament for preventing or treating a T1 inflammatory lung disease

Thus, given the general functional restraints on the immunogenicity of the constructs, the invention allows for all kinds of permutations of the origin IFNγ sequence, and all kinds of modifications therein.

Some of the IFNγ proteins of the pharmaceutical composition are sufficiently immunogenic, but for some the immune response will be enhanced if the composition further comprises an adjuvant substance. It is especially preferred to use an adjuvant which can be demonstrated to facilitate breaking of the autotolerance to autoantigens. Various methods of achieving adjuvant effect for the composition are known. General principles and methods are detailed in “The Theory and Practical Application of Adjuvants”, 1995, Duncan B. S. Stewart-Tull (ed.), John Wiley & Sons Ltd., and also in “Vaccines : New Generation Immunological Adjuvants”, 1995, Gregoriadis G et al. (eds.), Plenum Press. New York.

Preferred adjuvants facilitate uptake of the molecules by APCs, such as dendritic cells, and activate these. Non-limiting examples are selected from the group consisting of an immune targeting adjuvant; an immune modulating adjuvant such as a toxin, a cytokine, and a mycobacterial derivative; an oil formulation ; a polymer, a micelle forming adjuvant; a saponin; an immunostimulating complex matrix (ISCOM matrix); a particle; DDA aluminium adjuvants; DNA adjuvants; y-inulin; and an encapsulating adjuvant Details relating to composition and use of immunostimulating complexes can e.g. be found in the above-mentioned text-books dealing with adjuvants, but also Morein B. et al., 1995, as well as Barr I. G. and Mitchell G. F., 1996, provide useful instructions for the preparation of complete immunostimulating complexes. The current invention thus also relates to the use of a pharmaceutical composition comprising immunogenic IFNγ and an adjuvant for treating T1 inflammatory lung disease. More specifically, the current invention relates to the use of a pharmaceutical composition comprising autologous IFNγ and an adjuvant for treating T1 inflammatory lung disease.

Another approach of immunization includes the technology of nucleic acid immunization, also known as “genetic immunization”, “gene immunization” and “DNA vaccination”. The present invention thus also relates to the use of a DNA vaccine for preventing or treating T1 inflammatory lung disease. In this embodiment, the introduced nucleic acid encoding the modified IFNγ protein as described herein is preferably DNA which can be in the form of naked DNA, DNA formulated with charged or uncharged lipids, DNA formulated in liposomes, emulsified DNA, DNA included in a viral vector, DNA formulated with a transfection-facilitating protein or polypeptide, DNA formulated with a targeting protein or polypeptide, DNA formulated with calcium precipitating agents, DNA coupled to an inert carrier molecule, and DNA formulated with an adjuvant. In this context it is noted that practically all considerations pertaining to the use of adjuvants in traditional vaccine formulation apply to the formulation of DNA vaccines. Hence, all disclosures herein which relate to use of adjuvants in the context of protein or (poly)peptide based pharmaceutical compositions apply mutatis mutandis to their use in nucleic acid vaccination technology. The same holds true for other considerations relating to formulation and mode and route of administration and, hence, also these considerations discussed herein in connection with a traditional pharmaceutical composition apply mutatis mutandis to their use in nucleic acid vaccination technology.

Accordingly, the present invention also relates to a method of preventing or treating Tγ inflammatory lung disease by immunization with a pharmaceutical composition comprising a nucleic acid sequence encoding a immunogenic IFNγ protein and/or IFNγ-derived (poly)peptide. More specifically, the present invention relates to the use of a nucleic acid sequence encoding an immunogenic IFNγ protein and/or IFNγ derived (poly)peptide for the manufacture of a medicament for preventing or treating a T1 inflammatory lung disease.

Under normal circumstances, the nucleic acid of the vaccine is introduced in the form of a vector wherein expression is under control of a viral promoter.

Therefore, also provided are an expression vector which comprises a polynucleotide of the herein described proteins or peptides and which is capable of expressing the respective proteins or peptides, a host cell comprising the expression vector and a method of producing and purifying herein described proteins or peptides, pharmaceutical compositions comprising the herein described proteins or peptides and a pharmaceutically acceptable carrier and/or adjuvants.

Detailed disclosures relating to the formulation and use of nucleic acid vaccines are available, e.g. by Donnelly J. J. et al., 1997 and 1997a.

Administration

The molecule, protein, composition or agent of the current invention may be administered in any manner which is medically acceptable. In addition, it can at any time be administered together, simultaneously or sequentially, with another separate substance, molecule, antibody, composition, or a pharmaceutically and immunologically acceptable carrier and/or vehicle and/or an adjuvant.

Any of the conventional methods for administration of a pharmaceutical composition are applicable e.g parenterally, orally or by either subcutaneous, intradermal, subdermal, intravenous, intaarterial or intramuscular injection. Other modes of administration include suppositories and, in some cases, buccal, sublinqual, intraperitoneal, intravaginal, anal, and intracranial applications. Depending on the specific circumstances, local or systemic administration may be desirable.

One skilled in the art of preparing formulations can readily select the proper form and mode of administration depending upon the particular characteristics of the molecule, protein, composition or agent selected, the disease state to be treated, the stage of the disease, and other relevant circumstance.

According to the specific case, the “pharmaceutically effective amount” or “amount effective” is one that is sufficient to produce the desired effect. This can be monitored using several end-points known to those skilled in the art such as, but not limited to, mortality, morbidity, and the like. According to the specific case, the pharmaceutically effective amount should be determined as being the amount sufficient to cure the recipient in need of treatment, to prevent or at least to partially reduce or halt the disease or injury and its complications. The term “recipient” is intended to include living organisms, e.g. mammals, primates, and more specifically humans. Amounts effective for such use will depend on the severity of the disease and the general state of the recipient's health As such, dosage of the administered molecule, protein, composition or agent will vary depending upon such factors as the recipient's age, weight, height, sex, general medical condition, previous medical history, concurrent treatment with other pharmaceuticals, etc. Administration can be as a single dose or repeated doses one or more times after a specific period. When administering by injection, the administration may be by continuous injection, or by single or multiple boluses. Typically, such compositions are prepared as injectables either as liquid solutions or suspensions; solid forms suitable for solution in, or suspension in liquid prior to injection may also be prepared. The preparation may also be emulsified.

The active ingredient of the pharmaceutical composition is often mixed with excipients or carriers which are pharmaceutically acceptable and compatible with the active ingredient. Such excipients are inherently nontoxic and nontherapeutic. Examples of such excipients are water, saline, glycerol, ethanol, Ringer's solution, dextrose solution and Hank's solution, and/or combinations thereof. Nonaqueous excipients such as fixed oils and ethyl oleate may also be used. A preferred excipient is 5% dextrose in saline. The excipient may contain minor amounts of additives such as substances that enhance isotonicity and chemical stability, including buffers and preservatives. In addition, if desired, the composition may contain minor amounts of auxiliary substances such as wetting or emulsifying agents, pH buffering agents, or adjuvants which enhance the effectiveness of the composition. Examples of adjuvants are sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates, and agents for the adjustment of tonicity such as sodium chloride or dextrose.

### LEGEND TO THE FIGURE

FIG. 1: TNF-alfa levels were measured in the serum of the animals after 6 weeks of smoking. Sera from 3 placebo-treated animals and from 3 animals from the anti-IFNgamma treated group were tested.TNF-alfa was measured using an in house developed ELISA. The detection limit is 4 pg/ml. As shown in this figure, treatment of the smoking animals with anti-IFNgamma inhibits the TNF-alfa production.

### EXAMPLES

### Example 1:

Measurement of IFNγ in Lungs of Patients With T1 Inflammation

Immunological inflammation may be of a Type 1 with predominance of interleukin 2 (IL2) and interferon gamma (IFNγ) production or a Type 2 characterized by predominant IL-4 and IL-5 production. Presence of IFNγ in the lungs of patients with T1 inflammatory lung diseases can be measured in different ways as outlined hereunder.

One method is the measurement of cytokine levels in spontaneously produced and/or induced sputum. Sputum is defined as expectorations of fluid, cells and solutes that are present in the lining fluid of the upper bronchial tree. Spontaneous sputum can be obtained in a simple and non-invasive way whilst induced sputum is induced by exposure of individuals to a nebulised saline solution (Out et al. 2001). Protease inhibitors can be added shortly after isolation.

The gel and sol phase in sputum can be separated from each other by means of ultacentrifugation (50.000 g; 4° C.). Otherwise, mucus can be homogenized by treatment with dithiotreitol (DTT) (Karpati et al., 2000). Additional treatment by DNAse to degrade any DNA present or treatment with trypsin-EDTA may be necessary. Further low speed centrifugation (300 g) of homogenised sputum allows the separation of supernatant from the cellular compartment. Supernatant can be used as such or concentrated and eventually stored at −70° C. until assessment of cytokine levels.

BAL fluid is obtained by standard lavage protocol consisting of infusing 20 ml aliquots of sterile saline solution through an aspiration port followed by lavage collection through same. This procedure is repeated 5 times.

Sputum and/or BAL is collected from severe COPD patients (FEV1<30% predicted according to the GOLD criteria), moderate COPD patients (30% ≦FEV1<80% predicted according to the GOLD criteria), pack-year matched healthy smokers (normal FEV1 and no chronic symptoms of cough or sputum production) and control non-smokers. Patients are well characterized age, gender, pack-years smoking history, lung function test, acute exacerbations, inflammation.

Fluid phase of sputum or BAL is evaluated for presence of IFNγ by means of the Enzyme Linked Immunosorbent Assay (ELISA) method. A proteolytic environment like sputum and the treatment of sputum with reducing agents (DTT) may affect antigenic determinants as well as the characteristics of the antibodies and thus the performance of the immunoassays. Therefore, a particular assay has to be validated for its application in sputum. Commercially available kits from Medgenix or R&D Systems or Amersham Biosciences can be used to determine IFNγ immunoreactivities (Keatings, 2002).

Cellular fractions can be assessed for the presence of relative proportions of different cell types by Wright stain on cytospin slides. Cellular fractions can further be analysed for IFNγ production, by, immunohistochemistry on cytospin slides or RT-PCR on total cell RNA or by FACS analysis of intracelluar cytokines.

The leakage of plasma proteins (e.g. albumin, fibrinogen) from the blood into the airspace nay be considered as an overall marker for inflammation.

Presence of IFNγ can also be assessed in biopsies of smokers, non-smokers or ex-smokers with mild COPD or in lung fragments obtained by lobectomie of severe COPD patients. IFNγ can be evaluated by immunohistochemistry or RT-PCR.

### Example 2:

Effect of Blocking Ifn-gamma, by Anti-mouse Ifn-gamma Mab, on Cigarette Smoke Induced Emphysema in Mice

Introduction

Chronic Obstructive Pulmonary Disease (COPD) is characterized by the progressive development of a not fully reversible airflow limitation (Pauwels et al., 2001). The airflow limitation is due to a variable mixture of respiratory bronchiolitis and emphysema. A major risk factor for COPD is cigarette smoking. Inflammation of the airways and the lungs is thought to play a major role in the pathogenesis of COPD. Human studies clearly illustrate a smoking-induced inflammatory response, comprised of neutrophils, macrophages, dendritic cells, eosinophils, CD4⁺ and CD8⁺ T-lymphocytes in smokers' airways and lung parenchyma (Retamales et al., 2001; Casolaro et al., 1988). The exact role of individual inflammatory cells and mediators is unknown at the moment.

Wang et al. (2000) demonstrated that interferon gamma (IFN-gamma) causes emphysema with alveolar enlargement, enhanced lung volumes, enhanced pulmonary compliance, and macrophage- and neutropbil-rich inflammation when inducibly targeted, in a transgenic fashion, to the adult murine lung. Prominent protease and antiprotease aerations were also noted in these mice. They included the induction and activation of matrix metalloproteinase (MMP)-12 and cathepsins B, H, D, S, and L, the elaboration of MB-9, and the selective inhibition of secretory leukocyte proteinase inhibitor.

The aim of the current study is primary to investigate the effect of blocking IFN-gamma on the development of emphysema in mice exposed for six months to cigarette smoke. A secondary objective is to study the effect of anti-mouse IFN-gamma mAb on the cigarette smoke induced pulmonary inflammation.

Design of the Study

Three groups of 15 mice are followed for 24 weeks.

Group I is exposed to room air and treated twice a week (Monday and Thursday) intraperitoneally with vehicle.

Group II is exposed to cigarette smoke 5 days a week and treated twice a week (Monday and Thursday) intraperitoneally with vehicle.

Group III is exposed to cigarette smoke 5 days a week and treated twice a week (Monday and Thursday) intraperitoneally with 100 μg anti-mouse IFN-gamma mAb, F3.

Blood samples (300-400μl) are taken weekly Thursday) from the ocular sinus. Serum is prepared from each sample. Serum concentrations of anti-IFN-gamma mAb F3. IFN-gamma IL-6. TNF alfa IL-8. IL-16, different chemokines and other parameters, are analyzed using ELISA technology. NO was measured using Colormetric Nitric Oxide detection kit.

Immediately following the last exposure, animals are sacrificed and emphysema and lung inflammation are quantified using the following parameters:

Emphysema: Mean Linear Intercept, Mean Alveolar Surface, Mean number of alveolar wall breaks/alveolar space.

Inflammation:

BAL fluid: total number of cells, differential cell count for macrophages/monocytes, neutrophils, eosinophils and lymphocytes.

Lung tissue (single cell suspension): total number of cells, macrophages, dendritic cells, lymphocytes (CD3+), CD4+lymphocytes, CD8+lymphocytes, activated CD4+lymphocytes, activated CD8+lymphocytes.

Methods

Animals

Male C57B1/6 mice, 10 wks old (at the start of the experiment i.e. pre-bleeding), are purchased from Harlan (Zeist, The Netherlands). The mice are kept in standard animal research facilities and receive food and water ad libitum. Mice are kept in groups of 6. The local Ethics Committee approved all in vivo manipulations.

Experimental Design

Groups of 5 mice are exposed to the tobacco smoke of 5 cigarettes (Reference Cigarette 1R3, University of Kentucky, Lexington) per exposure. There ate 4 exposures a day with a 30 minutes smoke-free interval between each exposure. The animals smoke 5 days a week for up to 24 weeks. The control group is exposed to air. At week 24, mice are sacrificed and inflammatory parameters are examined in bronchoalveolar lavage (BAL) fluid and lung single cell suspension. Besides, histological evaluation of lung parenchyma is performed.

Tobacco Smoke Chamber

Mice are exposed to cigarette smoke, with the use of a smoking apparatus (S. Shapiro, Washington University Medical Center, USA) with the chamber adapted for a group of 6 mice (chamber volume of 7500 cm³). An optimal smoke:air ratio of 1:12 is obtained by injecting smoke and pressurized air at a flow rate of 200 ml/min and 2.4 L/min respectively.

Bronchoalveolar Lavage

Animals are sacrificed after i.p. injection of an overdose of pentobarbital (Sanofi, Libourne, France) and the trachea is surgically exposed and cannulated to perform bronchoalveolar lavage. 1 ml of Hank's balanced salt solution (HBSS) (GIBCO BRL), free of ionized calcium and magnesium, and supplemented with 0.05 mM EDTA (Sigma) is instilled 3 times via the tracheal cannula and recovered by gentle manual aspiration. The recovered bronchoalveolar lavage fluid (BAL) is centrifuged, the cell pellet is washed twice and finally resuspended in 1 ml of HBSS. A total cell count is performed in a Bürcker chamber and the differential cell counts (on at least 400 cells) are performed on cytocentrifuged preparations after May-Grünwald-Giemsa staining. Flow cytometric analysis of BAL-cells is also performed (see below).

The supernatant of the BAL is stored at −80° C. for measurement of IFN-gamma

Buffers and Media for Preparation of Single Cell Suspensions and Immunofluorescent Labeling

Digestion medium consists of RPMI 1640 supplemented with 2 mM L-glutamine, 10 μg/ml streptomycine, 100 U/ml penicilliin, 5% FCS, 0.001% β-mercaptoethanol (all from GIBCO BRL), 1 mg/ml collagenase type 2 (Worthington Biochemical Corp.), and 0.02 mg/ml DNase I (grade II from bovine pancreas; Boehringer). FACS-EDTA buffer contains PBS (GIBCO BRL) without Ca²⁺ or Mg²⁺, 0.1% azide, 5% EDTA-treated FCS, and 5 mM EDTA. EDTA-treated FCS is prepared by passing FCS though a 0.2 μm filter and mixing 1 ml of a 0.1 M disodium EDTA solution with 10 ml of filtered FCS.

Preparation of Lung Single Cell Suspensions

Following BAL the pulmonary and systemic circulation is perfused with saline containing 5 mM EDTA to remove the intravascular pool of cells. One lung is used for histology, the other is used for the preparation of a cell suspension. The lung is thoroughly minced using iridectomy scissors and incubated for 30 min in digestion medium in a humidified incubator at 37 ° C. and 5% CO₂. Organ fragments are resuspended, fresh digestion medium is added, and incubation is extended for another 15 min. After a final resuspension, very few organ debris are left. Samples are centrifuged and resuspended in calcium and magnesium-free PBS containing 10 mM EDTA at room temperature. Finally, the cells are subjected to RBC lysis, washed in FACS-EDTA, passed through a 50 μm cell stainer, and kept on ice until labeling. Cell counting is performed with a Z2 Beckman-Coulter particle counter (Beckman-Coulter).

Labeling of BAL-cells and Lung Single Cell Suspensions for Flow Cytometry

Cells are pre-incubated with Fc-receptor blocking antibody (anti-CD16/CD32, clone 2.4G2) to reduce non-specific binding. Monoclonal antibodies used to identify mouse DC populations are: biotinylated anti-CD11c (N418) and PE-conjugated anti-IA^{b} (AF6-120.1), followed by streptavidine-allophycocyanine (Sav-APC) (all from BD PharMingen). Isotype controls are PE-conjungated rat IgG_{2a,K}, rat IgG_{2b} and armenian hamster IgG_{2,K}. The following antibodies are used to stain mouse T-cell subpopulations: FITC-conjugated anti-CD4 (L3T4), FITC-conjugated anti-CD8 (Ly-2), and biotinylated anti-CD3 (145-2C11) monoclonal antibodies. The additional marker used for activation is anti-CD69 (H1.2F3). Biotinylated anti-CD3 is revealed by incubation with Sav-APC (all from BD Pharmingen). Monocytes/macrophages will be identified using FSC/SSC and CD11c staining.

As a last step before analysis cells are incubated with 7-amino-actinomycin (7-AAD or viaprobe, BD Pharmingen) for dead cell exclusion. All labeling reactions are performed on ice in FACS-EDTA buffer.

Flow Cytometry data acquisition is performed on a dual-laser FACS Vantage™ flow cytometer running CELLQuest™ software (Becton Dickinson). FlowJo software (www.Treestar.com) is used for data analysis.

Histology

After clamping the main bronchus of the lung excised for the preparation of a cell suspension, fixative (4% paraformaldehyde in PBS) is gently infused through the tracheal cannula by a continuous-release pump under pressure and volume controlled conditions (12 ml/hour, 3 psi, 10 min/lung). The lung is resected and fixed for an additional 4 h. After routine paraffin embedding, 3 μm sections are stained with hematoxylin and eosin (H&E) (Klinipath) and examined by light microscopy for histological changes.

Quantification of emphysema is performed in a blinded fashion using a Zeiss KS400 Image Analyzer system running a custom-made morphometry program The mean linear intercept (Lₘ) is measured for each mouse from 10 random fields by means of a 100×100 μm grid [15]. The mean alveolar surface (Aₘ) is also measured in 10 random fields per mouse. The number of alveolar wall breaks is counted per field and divided by the number of alveolar spaces in that field. The mean number of breaks/alveolar space is calculated from 10 random fields per mouse.

Results:

1. Effect of Anti-IFN-gammma mAb Treatment on Circulating TNF-alfa Levels Induced in Smoking Animals

Proinflammatory cytokines, particularly IL-1beta and TNF, may amplify the inflammatory response in COPD and be linked to disease severity. TNF-alfa is present in high concentrations in the sputum of COPD patients. Serum concentration of TNF-alfa are increased in weight-losing COPD patients, suggesting that it may play a role in the cachexia of severe COPD. TNF-alfa inhibits the expression of skeletal muscle proteins via activation of NF-kB. This suggests that inhibitors of TNF-alfa might be useful in reversing the skeletal wasting seen in COPD as well as reducing the airway inflammatory response (Barnes, 2003; Oudijk, 2003)

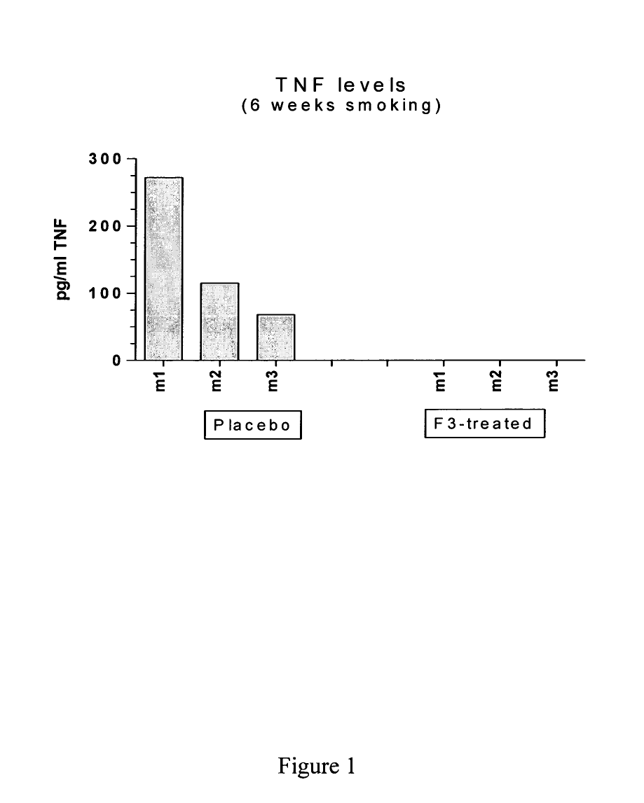

In our experiment, inflammation in the smoking animals is evidenced by increased levels of TNF-alfa in the circulation of the smoking mice, as compared to the non-smoking animals. TNF alfa is detectable in the smoking animals after 3 to 4 weeks of smoking. TNF-alfa levels are decreased by treatment of the animals with anti-IFNgamma mAb, as shown in FIG. 1.

Our results demonstrate that blocking IFNgamma has an effect on the systemic levels of TNF-alfa. Thus, these data demonstrate that blocking IFN-gamma has an effect on the inflammatory response induced by smoking in these animals.

### REFERENCES

Aaron S D, Angel J B, Lunau M, et al. 2001. Granulocyte inflammatory markers and airway infection during acute exacerbation of chronic obstructive pulmonary disease. Am. J. Resp. Crit. Care Med 163:349-55
Alexander J., Sidney J., Southwood S., Ruppert J., Oseroff C., Maewal A., Snoke K., Serra H M., Kubo R T., Sette A., et al. (1994). Development of high potency universal DR-restricted helper epitopes by modification of high affinity DR-blocking peptides. Immunity, 1(9): 751-761.
Ausubel et al. (1987) (1992) (1993) Current Protocols in Molecular Biology. Wiley Interscience, N.Y.
Balbi B., Di Stefano A., Capelli A., Balbo P., Lusuardi M., Ioli F. and Donner C. F. T1 and T2 cytokines in bronchoalveolar lavage of patients with COPD and with asthma. International Conference of the American Thoracic Society, 2002.
Barr I. G. and Mitchell G. F. (1996). ISCOMs (immunostimulating complexes): the first decade. Immunol. and Cell Biol. 74: 8-25.
Barnes P. J. (2000). Chronic Obstructive Pulmonary Disease. NEJM 343: 269-80.
Barnes P. J. (2001). New treatments for chronic obstructive pulmonary disease. Curr Opin Pharmacology, 1: 217-222.
Barnes P. J. (2002). New treatments for COPD. Nature reviews Drug discovery, 1: 437-446.
Barnes P J. (2003). New concepts in chronic obstructive pulmonary disease. Annu. Rev. Med., 54:113-29.
Baughman R. P. (2002). Therapeutic options for sarcoidosis: new and old. Curr Opin Pulm Med, 8: 464-469.
Billiau A. (1996) Interferon-γ: biology and role in pathogenesis. Advances in Immunology 62: 61-130.
Campbell A. M. (1984) Monoclonal Antibody Technology Techniques in Biochemistry and Molecular Biology, Elsevier Science Publishers, Amsterdam, The Netherlands.
Chackerian B., Lenz P., Lowy D. R. and Schiller J. T. (2002). Determinants of autoantibody induction by conjugated Papillomavirus virus-like particles. J Immunol, 169: 6120-6126.
Ciapponi L., Maione D., Scoumanne A., Costa P., Hansen M. B., Svenson M., Bendtzen K., Alonzi T., Paonessa G., Cortese R., Ciliberto G. and Savino R. (1997). Induction of interleukin-6 (IL-6) autoantibodies through vaccination with an engineered IL-6 receptor antagonist. Nature Biotechnol, 15: 997-1001.
Coligan J. E., Kruisbeek A. M., D. H. Margulies D. H. (1992) (1993) Current Protocols in Immunology, Green Publishing Association and Wiley Interscience, N.Y.
Contreras C. E., Ploton I. N., Siliciano R. F., Karp C. L., Viscidi R. and Kumar N. (1998). Mapping of specific and promiscuous HLA-DR-restricted T-cell epitopes on the Plasmodium falciparum 27-kilodalton sexual stage-specific antigen Infect Immun, 66(8): 3579-90.
Casolaro M A, Bernaudin J F, Saltini C, Ferrans V J, Crystal R G.(1988). Accumulation of Langerhans' cells on the epithelial surface of the lower respiratory tract in normal subjects in association with cigarette smoking. Am Rev Respir Dis, 137:406-11.
Dalum I., Jensen M. R., Hindersson P., Elsner H. I., Mouritsen S. (1996). Breaking of B cell tolerance toward a highly conserved self protein. J Immunol 157(11): 4796-804.
Dalum I., Butler D. M., Jensen M. R., Hindersson P., Steinaa L., Waterston A. M., Grell S. N., Feldmann M., Elsner H. I. and Mouritsen S. (1999). Therapeutic antibodies elicited by immmunization against TNF-α. Nature Biotechnol, 17: 666-669.
deBoer W. I. (2002). Cytokines and therapy in COPD. CHEST, 121:209S-218S.
Donnelly J. J., Ulmer J. B., and Liu M. A. (1997a). DNA vaccines. Life Sci., 60(3): 163-172.
Donnelly J. J., Ulmer J. B., Shiver J. W. and Liu M. A. (1997). DNA vaccines. Annu. Rev. Immunol., 15: 617-648.
Doolan D. L., Southwood S., Chesnut R., Appela E., Gomez E., Richards A., Higashimoto Y. I., Maewal A., Sidney J., Gramzinski R. A., Mason C., Koech D., Hoffman S. L. and Sette A. (2000). HLA-DR-promiscuous T cell epitopes from Plasmodium falciparum pre-erythrocytic-stage antigens restricted by multiple HLA alleles. J Immunol, 165:1123-1137.
Dustin M. L., Singer K. H., Tuck D. T. and T. A. Springer (1988) Adhesion of T lymphoblasts to epidermal keratinocytes is regulated by interferon-γ and is mediated by intercellular adhesion molecule 1 (ICAM-1). J. Exp. Med. 167: 1323-1340.
Fernando G. J., Tindle R. W. and Frazer I. H. (1995). T-helper epitopes of the E7 transforming protein of cervical cancer associated human papillomavirus type 18 (HPV18). Virus Res, 36(1):1-13.
Freedman A. S., Freeman G. J., Rhynhart K. and L. M. Nadler (1991) Selective induction of B7/BB-1 on interferon-gamma stimulated monocytes: a potential mechanism for amplification of T cell activation through the CD28 pathway. Cell. Immunol 137:429-437.
Friedl-Hajek R., Spangfort M. D., Schou C., Breiteneder H., Yssel H. and Joost van Neerven R. J. (1999). Identification of a highly promiscuous and an HLA-allele specific T-cell epitope in the birch major allergen Bet v 1: HLA restriction, epitope mapping and TCR sequence comparisons. Clin Exp Allergy, 29(4): 478-87.
Gaudebout P., Zeliszewski D., Golvano J. J., Pignal C., Le Gac S., Borras-Cuesta F. and Sterkers G. (1997). Binding analysis of 95 HIV gp120 peptides to HLA-DR1101 and -DR0401 evidenced many HLA-class II binding regions on gp120 and suggested several promiscuous regions. J Acquir Immune Defic Syndr Hum Retrovirol, 14(2): 91-101.
Gish W. and States D. J. (1993). Identification of protein coding regions by database similarity search. Nature Genet, 3:266-272.
Gonzalez S., Alvarez A., Caballero E., Vina L., Guillen G. and Silva R. (2000). P64k meningococcal protein as immunological carrier for week immunogens. Scand J Immunol, 52(2): 113-116.
Harlow E., David L. et. al. (1988) Antibodies: A Laboratory Manual, Cold Spring Harbor Publications.
Hautamaki R. D., Kobayashi D. K., Senior R. M. and Shapiro S. D. (1997). Requirement for macrophage elastase for cigarette smoke-induced emphysema in mice. Science, 277(5334):2002-4.
Holliger P., T. Prospero, and G. Winter (1993) Diabodies: small bivalent and bispecific antibody fragments. Proc. Natl. Acad. Sci. USA 90(14):6444.
Hurle M. R. and M. Gross (1994) Protein engineering techniques for antibody humanization. Curr. Opin. Biotech 5:428-433.
Iliades P., Kortt A. A., and P. Hudson (1997) Triabodies: single chain Fv fragments without linker form trivalent trimers. FEBS Letters 409:437-441.
Kaplan N. M., Palmer B. F. and Weissler J. C. (2000). Syndromes of Severe Asthma Am Med Sci, 319(3): 166-176.
Karlsson R., Michaelsson A. and Mattsson L. (1991). Kinetic analysis of monoclonal antibody-antigen interactions with a new biosensor based analytical system. J Immunol Methods, 145: 129-240.
Keatings V M, Collins P D, Scott D M, Barnes P J. 1996. Differences in interleukin-8 and tumor necrosis factor-α in induced sputum from patients with chronic obstructive pulmonary disease or asthma. Am. J. Resp. Crit. Care Med. 153:530-34
Kilgus J., Jardetzky T., Gorga J. C., Trzeciak A., Gillessen D. and Sinigaglia F. (1991). Analysis of the permissive association of a malaria T cell epitope with DR molecules. J Immunol, 146(1): 307-15.
Kim S. K., Ragupathi G., Capello S., Kagan E., and Livingston P. O. (2001). Effect of immunological adjuvant combinations on the antibody and T cell repsonse to vaccination with MUC1-KLH and GD3-KLH conjugates. Vaccine, 19: 530-537.
Kim Y -M. and Son K. (1996). A nitric oxide production assay for interferonγ. J Immunol Methods, 198:203-209.
Kobayashi H., Wood M., Song Y. Appella E. and Celis E. (2000). Defining promiscuous MHC class II helper T-cell epitopes for the HER2/neu tumor antigen. Cancer Res, 60(18): 5228-36.
Kortt A., Lab M., Oddie G., Gruen C., Burns J., Pearce L., Atwell J., McCoy A., Howlet G., Metzger D., Webster R. and P. Hudson (1997). Single-chain Fv fragments of anti-neuramidase antibody NC10 containing five- and ten-residue linkers form dimers and with zero-residue linker a trimer. Prot. Eng. 10:423-.
Köhler G. and C. Milstein (1975) Continuous cultures of fused cells secreting antibody of predefined specificity. Nature 256:495.
Launois P., Deleys R., Niang M. N., Drowart A., Andrien M., Dierckx P., Cartel J. L., Sarthou J. L., Van Vooren J. P. and Hyugen K. (1994). T-cell-epitope mapping of the major secreted mycobacterial antigen Ag85A in tuberculosis and leprosy. Infect Immun, 62(9): 3679-87.
Leckie M. J., Bryan S. A., Hansel T. T. and Barnes P. J. (2000). Novel therapy for COPD. Exp Opin Invest Drugs, 9: 3-23.
Lethbridge M. W. G, Allenby M. I., Jensen M. W., Kemeny D. M. and O'Connor B. J. (2002). Airway T lymphocytes expressing IFN-γ are reduced in smokers with chronic obstructive pulmonary disease (COPD). Annual Congress 2002 of the European Respiratory Society-Stockholm.
Lewis J. A. (1995). A sensitive bioassay for interferons. J Immumol Methods, 185: 9-17.
Li Y., Wang M -N., Li H., King K. D., Bassi R., Sun H., Santiago A, Hooper A. T., Bohlen P. and Hicklin D. J. (2002). Active immunization against the vascular endothelial growth factor receptor flk1 inhibits tumor angiogenesis and metastasis. J Exp Med, 195(12): 1575-1584.
Lorsbach R. B., Murphy W. J., Lowenstein C. .J, Snyder S. H. and S. W. Russel (1993) Expression of the nitric oxide synthase gene in mouse macrophages activated for tumor cell killing. J. Biol. Chem. 268: 1908-1913.
Lowenadler B. and Lycke N. (1994). Fusion proteins with heterologous T helper epitopes. Recombinant E. coli heat-stable enterotoxin proteins. Int Rev Immunol, 11(2): 103-11.
Majori M., Corradi M., Caminati A., Giancarlo C., Bertacco S. and Pesci A. (1999). Predominant TH1 cytokine pattern in peripheral blood from patients with COPD. J Allergy Clin Immunol, 103:458-62.
Mannino D. M. (2002). COPD: Epidemiology, prevalence, morbidity and mortality, and disease heterogeneteity. CHEST, 121: 121S-126S.
Malmqvist M. (1999). BIACORE: an affinity biosensor system for characterization of biomolecular interactions. Biochemical Society Transactions, Vol 27.
Meager A. (2002). Biological assays for interferons. J Immunol Methods, 261 (1-2): 21-36.
Mendez M. J., Green L. L., Corvalan J. R., Jia X. C., Maynard-Currie C. E., Yang X. D., Gallo M. L., Louie D. M., Lee D. V., Erickson K. L., Luna J., Roy C. M., Abderrahim H., Kirshenbaum F., Noguchi M., Smith D. H., Fukushima A., Hales J. F., Klapholz S., Finer M. H., Davis C. G., Zsebo K. M. and Jakobovits A. (1997). Functional transplant of megabase human immunoglobulin loci recapitulates human antibody response in mice. Nat Genet, 15(2):146-56.
Möllers M., Aries S. P., Drömann D., Mascher B., Braun J. and Dalhoff K. (2001). Intracellular cytokine repertoire in different T cell subsets from patients with sarcoidosis. Thorax, 56: 487-493.
Morein B. et al. (1995). Clin Immunother. 3:461-475.
Muller R. (1983) Determination of affinity and specificity of anti-hapten antibodies by competitive radioimmunoassay. Methods Enzymol. 92: 589-601.
Mustafa A. S., Shaban F. A., Abal A. T., Al-attiyah R., Wiker H. G., Lundin K. E., Oftung F. and Huygen K. (2000). Identification and HLA restriction of naturally derived Th1-cell epitopes from the secreted Mycobacterium tuberculosis antigen 85B recognized by antigen-specific human CD4+ T-cell lines. Infect. Immun., July: 3933-3940.
NIH, National heart, lung and blood institute (1995). Sarcoidosis. NIH publication No. 95-3093, reprinted July 1995.
NIH, National heart, lung and blood institute (1997). The Lungs in health and disease. NIH Publication No. 97-3279, August 1997.
NHLBI/WHO. Executive Summary, Global Strategy for the diagnosis, management and prevention of chronic obstructive pulmonary disease. Workshop Report. Pace J. L., Russell S. W., Torres B. A., Johnson H. M. and P. W. Gray (1983) Recombinant mouse γ interferon induces the priming step in macrophage activation for tumor cell killing. J. Immunol. 130: 2011-2013.
Oudijk E -JD, Lammers J -W J, Koenderman L. (2003) Systemic inflammation in chronic obstructive pulmonary disease. Eur Respir J, 22(S46): 5s-13s
Papiris S., Kotanidou A., Malagar K. and Roussos C. (2002). Clinical review: severe asthma. Crit Care, 6(1): 3044.
Pauwels R A, Buist A S, Calverley P M, Jenkins C R, Hurd S S. (2001) Global strategy for the diagnosis, management, and prevention of chronic obstructive pulmonary disease. NHLBI/WHO Global Initiative for Chronic Obstructive Lung Disease (GOLD) Workshop summary. Am J Respir Crit Care Med, 163:1256-76.
Retamales I, Elliott W M, Meshi B, Coxson H O, Pare P D, Sciurba F C, et al. (2001). Amplification of inflammation in emphysema and its association with latent adenoviral infection. Amer J Respir Crit Care Med, 164: 469-473.
Poljak R. J. (1994) Production and structure of diabodies. Structure 2:1121-1123.
Richard M., Grencis R. K., Humphreys N. E., Renauld J -C. and Van Snick J. (2000). Anti-IL-9 vaccination prevents worm expulsion and blood eosinophilia in Trichuris muris-infected mice. PNAS, 97(2): 767-772.
Roguska M. A., Pedersen J. T., Keddy C. A., Henry A. H., Searle S. J., Lambert J. M., Goldmacher V. S., Blättler W. A., Rees A. R.; and B. C. Guild (1994) Humanization of murine monoclonal antibodies through variable domain resurfacing. Proc. Natl. Acad. Sci. USA 91: 969-973.
Rossman M. D. (2001). Chronic Beryllium Disease: A hypersensitivity disorder. Appl Occup Environm Hygiene, 16(5): 615-618.
Sad S., Rao K., Arora R., Talwar G. P. and Raghupathy R. (1992). Bypass of carrier-induced epitope specific suppression using a T-helper epitope. Immunology, 76: 599-603.
Sangiuolo F., D'Apice M. R., Bruscia E., Lucidi V. and Novelli G. (2002). Towards the pharmacogenomics of cystic fibrosis. Pharmacogenomics, 3(1): 75-87.
Saltini C. (2001). Beryllium Disease. Am J Med Sci, 321(1): 89-98.
Shigehara K., Shijubo N. Obmichi M., Tkahashi R., Kon S., Okamura H., Kurimoto M., Hiraga Y., Tatsumo T., Abe S. and Sato N. (2001). IL-12 and IL-18 are increased and stimulate IFN-γ production in sarcoid lungs. J Immunol 166: 642-649.
Snapper C. M. and W. E. Paul (1987) Interferon-γ and B cell stimulatory factor-1 reciprocally regulate Ig isotype production. Science 236: 944-947.
Steinman R. M., Noguiera N., Witmer M. D., Tydings J. G. and I. S. Mellman (1980) Lymphokine enhances the expression and synthesis of Ia antigens on cultural mouse peritoneal macrophages. J. exp. Med. 152: 1248-1261.
Stirling R. G. and Chung K. F. (2001). Severe Asthma: Definition and mechanisms. Allergy, 56: 825-840.
Storni T., Lechner F., Erdmann I., Bächi T., Jegerlehner A., Dumrese T., Kündig T. M., Ruedl C. and Bachmann M. F. (2002). Critical role for activation of antigen-presenting cells in priming of cytotoxic T cell responses after vaccination with virus-like particles. J Immunol, 168: 2880-2886.
Talwar G. P. (1999). Vaccines and passive immunological approaches for the control of fertility and hormone-dependent cancers. Immunological Reviews, 171: 173-192.
Udono H. and Srivastava P. K. (1993). Heat shock protein 70-associated peptides elicit specific cancer immunity. J Exp Med, 178(4): 1391-6.
Verneresson M., Ledin A., Johansson J. and Hellman L. (2002). Generation of therapeutic antibody responses against IgE through vaccination. FASEB J. 16: 875-877.
Wang Z., Zheng T., Zhu Z., Homer R. J., Riese R., Chapman H. A., Shapiro S. D. and Elias J. A. (2000). Interferon γ induction of pulmonary emphysema in the adult murine lung. J Exp Med, 192(11): 1587-1599.
Wenzel S. E., Szefler S. J., Leung D. Y., Sloan S. I., Rex M. D. and Martin R. J. (1997). Bronchoscopic evaluation of severe asthma. Persistent inflammation associated with high dose glucocorticoids. Am J Respir Crit Care Med, 156(3 Pt 1):737-43.
Zhu Z., Zapata G., Shalaby R., Snedecor B., Chen H. and P. Carter (1996) High level secretion of a humanized bispecific diabody from Escherichia coli. Biotechnology 14:192-196.

1. Use of an IFNγ neutralizing molecule for the manufacture of a medicament for preventing or treating a T1 inflammatory lung disease.

2. Use according to claim 1 wherein said T1 inflammatory lung disease is selected from the group consisting of COPD, emphysema, chronic bronchitis, bronchiolitis, severe asthma, sarcoidosis, berylliosis and cystic fibrosis.

3. Use according to claims 1-2, wherein said molecule is an anti-IFNγ antibody or a fragment thereof.

4. Use according to claim 3, wherein said antibody is a monoclonal antibody.

5. Use according to claim 4, wherein said antibody is the antibody D9D10 or a fragment thereof.

6. Use according to claim 3, wherein said antibody is a humanized monoclonal antibody.

7. Use according to claim 6, wherein said antibody is a humanized D9D10 antibody.

8. Use of immunogenic IFNγ for the manufacture of a medicament for preventing or treating a T1 inflammatory lung disease.

9. Use according to claim 8 wherein said T1 inflammatory lung disease is selected from the group consisting of COPD, emphysema, chronic bronchitis, bronchiolitis, severe asthma, sarcoidosis, berylliosis and cystic fibrosis.

10. Use according to claim 8, wherein immunogenic IFNγ is a modified IFNγ.

11. Use according to claim 10, wherein the IFNγ protein is modified to immunogenic IFNγ by crosslinking IFNγ to an immunogenic non-self carrier protein.

12. Use according to claim 10, wherein the IFNγ protein is modified to immunogenic IFNγ by introducing at least one foreign T-cell epitope by insertion and/or substitution and/or addition and/or conjugation.

13. Use according to claim 8, wherein immunogenic IFNγ is a non-self IFNγ administered with or without adjuvant.

14. Use according to claim 8, wherein immunogenic IFNγ is autologous IFNγ in combination with an adjuvant.

15. Use of at least one autologous antigen presenting cell loaded with IFNγ for the manufacture of a medicament for preventing or treating a T1 inflammatory lung disease.

16. Use according to claim 16, wherein the antigen presenting cell is a dendritic cell.
